(19) United States
(12) Patent Application Publication
(10) Pub. No.: US 2025/0152079 A1
(43) Pub. Date: May 15, 2025

(54) APPARATUS AND METHOD FOR PRESENTING VISUAL STIMULUS BY USING AUGMENTED REALITY

(71) Applicants: HYUNDAI MOTOR COMPANY, Seoul (KR); Kia Corporation, Seoul (KR)

(72) Inventors: Hoseung CHA, Guri-si (KR); Seungkyu NAM, Seoul (KR); Taejun LEE, Suwon-si (KR)

(73) Assignees: HYUNDAI MOTOR COMPANY, Seoul (KR); Kia Corporation, Seoul (KR)

(21) Appl. No.: 18/678,729

(22) Filed: May 30, 2024

(30) Foreign Application Priority Data
Nov. 13, 2023 (KR) 10-2023-0156153

(51) Int. Cl. A61B 5/378; A61B 5/00; A61B 90/00

(52) U.S. Cl. CPC A61B 5/378; A61B 5/7264; A61B 90/36; A61B 2090/365

(57) ABSTRACT

An apparatus for presenting a visual stimulus by using augmented reality may include an augmented reality module configured to recognize a control target through an augmented reality (AR) glass, and to dispose the visual stimulus on the recognized control target, an electroencephalogram measurement module configured to measure an electroencephalogram of a user gazing at the visual stimulus, a visual stimulus detection module configured to analyze the measured electroencephalogram to detect a visual stimulus signal (VEP), and to process the detected visual stimulus signal to classify the visual stimulus and to identify the control target, and a control module configured to control the identified control target through a network.

### CROSS-REFERENCE TO RELATED APPLICATION

This application claims priority to and the benefit of Korean Patent Application No. 10-2023-0156153 filed in the Korean Intellectual Property Office on Nov. 13, 2023, the entire contents of which is incorporated herein by reference.

### TECHNICAL FIELD

The present disclosure relates to an apparatus and method for presenting a visual stimulus by using augmented reality. More particularly, the present disclosure relates to an apparatus and method for presenting a visual stimulus by using augmented reality for a brain-computer interface (BCI) system based on immersive augmented reality (AR).

### BACKGROUND

BCI refers to a technology that operates external devices using only thoughts, and mainly delivers desired commands by collecting and analyzing electroencephalogram signals. The electroencephalogram signal may be divided into an invasive electroencephalogram, which uses a sensor invaded into the scalp and a non-invasive electroencephalogram, which senses the electroencephalogram by attaching an electrode on the scalp surface, and for practicality, non-invasive BCI technology is mainly being researched. Particularly, it is well known that the steady-state visual evoked potential (SSVEP) generated when a visual stimulus flickering at a particular frequency is gazed at, or the P300 potential generated due to unpredicted visual stimuli, or the like may be observed in the electroencephalogram. Visually evoked potential-based BCI technologies such as SSVEP or P300 are mainly used as a means of communication for patients.

Recently, research for combining AR technology and BCI technology is being actively conducted due to the development of AR technology that may augment virtual information in real space at any time. There is an advantage in that BCI technology may be used without space restrictions as visual stimuli may be projected onto AR glasses without having to present them on a computer monitor as before.

Methods for presenting an SSVEP visual stimulus are mainly based on studies that simply borrow the 2D form of existing monitors. However, since existing studies use 2D visual stimuli as is, hindering users' natural AR interaction may be deteriorated.

### SUMMARY

The present disclosure attempts to provide an apparatus and method for presenting a visual stimulus by using augmented reality capable of synthesizing virtual steady-state visual evoked potential (SSVEP)-based visual stimuli to objects in the real world.

The present disclosure attempts to provide an apparatus and method for presenting a visual stimulus by using augmented reality capable of presenting a visual stimulus at an appropriate location in consideration of the surrounding environment and brain-computer interface (BCI) control target.

An apparatus for presenting a visual stimulus by using augmented reality may include an augmented reality module configured to recognize a control target through an augmented reality (AR) glass, and to dispose the visual stimulus on the recognized control target, an electroencephalogram measurement module configured to measure an electroencephalogram of a user gazing at the visual stimulus, a visual stimulus detection module configured to analyze the measured electroencephalogram to detect a visual stimulus signal (VEP), and to process the detected visual stimulus signal to classify the visual stimulus and to identify the control target, and a control module configured to control the identified control target through a network.

The augmented reality module may be configured to register an image of the control target received from database.

The augmented reality module may include a control target detector configured to detect a location of the control target gazed at by the user through the AR glass and to dispose a particular means to identify the control target.

The particular means may include a virtual outline disposed on a contour of the control target.

The augmented reality module may further include a visual stimulus arrangement unit configured to dispose a steady-state visual evoked potential (SSVEP)-based visual stimulus flickering at a particular frequency on the outline.

The visual stimulus arrangement unit may be configured to dispose an additional visual stimulus that provides an interface for controlling the control target to a particular location that does not overlap the outline of the identified control target.

The visual stimulus detection module may include an electroencephalogram signal receiver configured to receive an electroencephalogram signal, a visual stimulus signal detector configured to analyze the received electroencephalogram signal to detect the visual stimulus signal corresponding to a particular frequency, a feature extracting unit configured to extract a feature of the visual stimulus signal, and a visual stimulus classifier configured to classify the visual stimulus based on the extracted feature and to identify the control target.

The augmented reality module may be configured to detect locations of a plurality of control targets through the AR glass, dispose virtual outlines to contours of the plurality of control targets, respectively, and dispose VEP-based visual stimuli flickering at different frequencies on the virtual outlines, respectively, and the virtual outlines may include 3-dimensional outlines.

The visual stimulus detector may be configured to analyze similarity between the visual stimulus signal detected from the electroencephalogram of the user gazing at the visual stimulus having a particular frequency and a reference signal according to a predefined frequency to detect a frequency having a highest similarity, and identify the control target gazed at by the user based on the detected frequency.

The visual stimulus detection module may be configured to dispose an additional visual stimulus that provides an interface for an additional interaction after the control target is identified at a particular location that does not overlap the location of the control target.

A method for presenting a visual stimulus by using augmented reality may include recognizing a control target gazed at by a user through an augmented reality (AR) glass, and disposing the visual stimulus on the recognized control target, measuring an electroencephalogram of the user gazing at the visual stimulus, analyzing the measured electroencephalogram to detect a visual stimulus signal (VEP), and processing the detected visual stimulus signal to classify the visual stimulus and to identify the control target, and controlling the identified control target through a network.

The disposing the visual stimulus may include detecting a location of the control target in a 3-dimensional space and disposing a virtual outline to a 3-dimensional contour of the control target.

The disposing the visual stimulus may further include disposing a steady-state visual evoked potential (SSVEP)-based visual stimulus flickering at a particular frequency on the virtual outline.

The identifying the control target may include extracting a feature of the visual stimulus signal, and classifying the visual stimulus based on the extracted feature.

The disposing the visual stimulus may further include disposing an additional visual stimulus providing an interface for controlling the control target at a location that does not overlap the control target, when the control target is identified.

The disposing the visual stimulus may include detecting locations of a plurality of control targets in a 3-dimensional space to dispose a 3-dimensional virtual outline on each of contours of the plurality of control targets.

The disposing the visual stimulus may further include disposing the VEP-based visual stimulus flickering at different frequencies on the virtual outlines.

The identifying the control target may include analyzing similarity between a visual stimulus signal detected from the user gazing at the visual stimulus of a particular frequency visual stimuli having the different frequencies and a reference signal based on a predefined frequency to detect a frequency having a highest similarity, and identifying the control target gazed at by the user based on the detected frequency.

The disposing the visual stimulus may further include disposing an additional visual stimulus for controlling the identified control target on the 3-dimensional space so as not to overlap the identified control target.

The visual stimulus may include a checkerboard image inverted at a particular frequency.

An apparatus and method for presenting a visual stimulus by using augmented reality according to an embodiment may synthesize virtual steady-state visual evoked potential (SSVEP)-based visual stimuli to objects in the real world.

An apparatus and method for presenting a visual stimulus by using augmented reality according to an embodiment may provide natural augmented reality brain-computer interaction by present a visual stimulus at an appropriate location in consideration of the surrounding environment and brain-computer interface (BCI) control target.

### BRIEF DESCRIPTION OF THE DRAWINGS

FIG. 1 is a drawing showing a brain-computer interface (BCI) system including an apparatus for presenting a visual stimulus by using augmented reality according to an embodiment.

FIG. 2 is a block diagram of an apparatus for presenting a visual stimulus by using augmented reality according to an embodiment.

FIG. 3 is a drawing showing an operation process of an apparatus for presenting a visual stimulus by using augmented reality according to an embodiment.

FIG. 4 and FIG. 5 are flowcharts showing a method for presenting a visual stimulus by using augmented reality according to an embodiment.

FIG. 6 is an example diagram showing a process of presenting and controlling a visual stimulus with respect to a single control target according to an embodiment.

FIG. 7 is an example diagram showing a process of presenting and controlling a visual stimulus with respect to a plurality of control targets according to an embodiment.

FIG. 8 is drawing for explaining a computing device according to an embodiment.

### DETAILED DESCRIPTION OF THE EMBODIMENTS

An embodiment of the disclosure will be described more fully hereinafter with reference to the accompanying drawings such that a person skill in the art may easily implement the embodiment. As those skilled in the art would realize, the described embodiments may be modified in various different ways, all without departing from the spirit or scope of the present disclosure. In order to clarify the present disclosure, parts that are not related to the description will be omitted, and the same elements or equivalents are referred to with the same reference numerals throughout the specification.

In addition, unless explicitly described to the contrary, the word “comprise” and variations such as “comprises” or “comprising” will be understood to imply the inclusion of stated elements but not the exclusion of any other elements. Terms including an ordinary number, such as first and second, are used for describing various constituent elements, but the constituent elements are not limited by the terms. The terms are only used to differentiate one component from other components.

In addition, the terms “unit”, “part” or “portion”, “-er”, and “module” in the specification refer to a unit that processes at least one function or operation, which may be implemented by hardware, software, or a combination of hardware and software.

Hereinafter, embodiments of the present disclosure will be described with reference to the drawings.

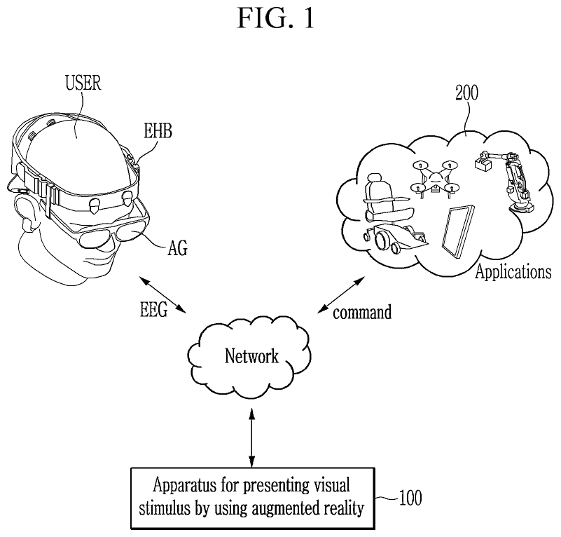

FIG. 1 is a drawing showing a brain-computer interface (BCI) system including an apparatus for presenting a visual stimulus by using augmented reality according to an embodiment. FIG. 1 illustrates an immersive augmented reality (AR) brain-computer interface (BCI) system according to an embodiment.

An apparatus 100 for presenting a visual stimulus by using augmented reality (hereinafter referred to as a visual stimulus presenting apparatus) proposes a new method for presenting a visual stimulus for the BCI system using an immersive augmented reality. A visual stimulus presenting apparatus 100 proposes a method of synthesizing virtual SSVEP visual stimuli to objects in the real world. That is, a visual stimulus presenting apparatus 100 may present, by synthesizing a virtual SSVEP visual stimulus to a control target 200, which is an object in the real world gazed at by the user through an AR glass AG.

A visual stimulus presenting apparatus 100 may analyze the electroencephalogram signal (EEG) received through an electroencephalogram measurement headband (EHB) attached with an electrode for measuring the electroencephalogram of the user gazing at the visual stimulus, to detect visual stimulus and to identify the control target 200.

A visual stimulus presenting apparatus 100 may communicate with the identified control target 200 through a network, and may perform a control such as providing an operation command according to the detected visual stimulus.

The control target 200 may include various external devices. The control target 200 may be a device capable of communication with a visual stimulus presenting apparatus 100 through Bluetooth, Wi-Fi, IoT, a cloud, or the like. For example, the control target 200 may include smart home appliances, robot arms, electric wheelchairs, or the like.

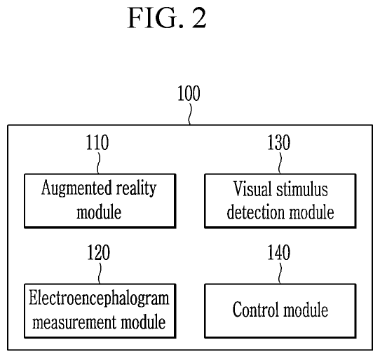

FIG. 2 is a block diagram of an apparatus for presenting a visual stimulus by using augmented reality according to an embodiment.

Referring to FIG. 2, an apparatus for presenting a visual stimulus by using augmented reality 100 may include an augmented reality module 110, an electroencephalogram measurement module 120, a visual stimulus detection module 130 and a control module 140.

The augmented reality module 110 may include an AR glass. The augmented reality module 110 may be connected to the AR glass through a wired and/or wireless network. The augmented reality module 110 may recognize a control target gazed at by user through the AR glass, and dispose or synthesize a visual stimulus to the recognized control target.

The augmented reality module 110 may detect a location of the control target that exists in the 3-dimensional real world. The augmented reality module 110 may detect a contour of the control target. The augmented reality module 110 may mark the control target based on the contour of the control target.

For example, the augmented reality module 110 may dispose an outline on the contour of the control target. The augmented reality module 110 may synthesize a 3-dimensional virtual outline onto the contour of the control target of the 3-dimensional real world.

The augmented reality module 110 may dispose the steady-state visual evoked potential (SSVEP)-based visual stimulus on the virtual outline. The visual stimulus may include a virtual image flickering at a particular frequency. The visual stimulus may include a checkerboard inverted according to the particular frequency.

The electroencephalogram measurement module 120 may measure the electroencephalogram of the user gazing at the visual stimulus. The electroencephalogram measurement module 120 may be connected to the electroencephalogram measurement headband wrapped around a user's head through a network. The electroencephalogram measurement module 120 may detect an EEG signal by measuring the electroencephalogram of the user through the electroencephalogram measurement headband. The electroencephalogram measurement module 120 may transfer the EEG signal to the visual stimulus detection module 130.

The visual stimulus detection module 130 may analyze the EEG signal to detect a visual stimulus signal. The visual stimulus signal may include a steady-state visual evoked potential (SSVEP). The visual stimulus detection module 130 may process the steady-state visual evoked potential to classify the visual stimuli.

The visual stimulus detection module 130 may classify the visual stimulus gazed at by the user to identify the control target gazed at by the user. The visual stimulus signal may include the electroencephalogram signal generated according to the visual stimulus, and may be referred to a visual stimulus evoked signal or a visual evoked signal.

The control module 140 may be connected to the visual stimulus detection module 130 through a network and may control the control target. That is, the control module 140 may perform a control with respect to the identified control target based on the detected visual stimulus.

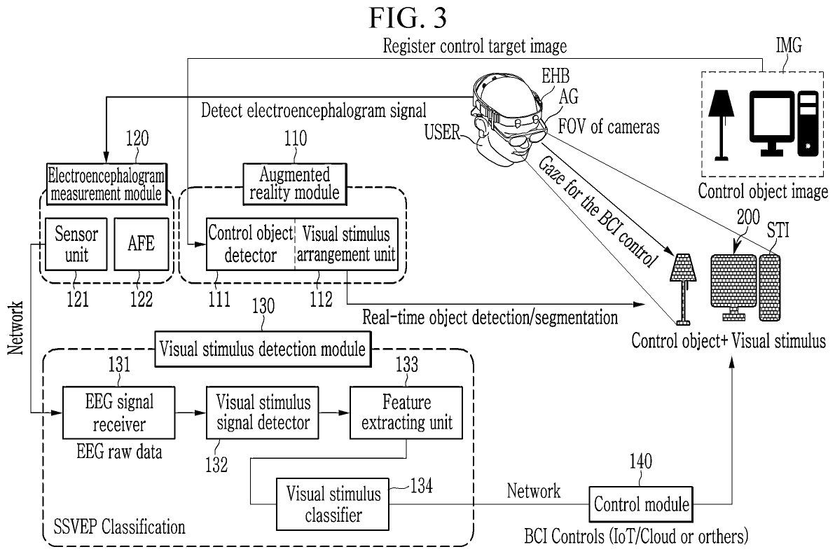

FIG. 3 is a drawing showing an operation process of an apparatus for presenting a visual stimulus by using augmented reality according to an embodiment.

In FIG. 3, among objects located within the field of view (FOV) of the user wearing the AR glass AG and detected by the gaze of the user, objects capable of communication with a visual stimulus presenting apparatus 100 (refer to FIG. 1) through a network, may correspond to the control target 200. For example, smart lighting devices, smart monitors, smart computers, and the like may correspond to the control target 200. A visual stimulus presenting apparatus 100 may synthesize a virtual SSVEP-based visual stimulus STI on the control targets 200.

The augmented reality module 110 may receive and register the control target images IMG from database. The control target images IMG may include image information with respect to the control targets 200 to be controlled through a visual stimulus presenting apparatus 100. The control target images IMG may be used to generate the virtual visual stimulus STI. The control target images IMG may be used to generate the virtual outline.

The augmented reality module 110 may include the control target detector 111 and a visual stimulus arrangement unit 112.

The control target detector 111 may detect a location of the control target 200 gazed at by user through the AR glass AG, and may dispose an identifying means for identifying the control target to the control target. For example, the control target detector 111 may dispose the virtual outline to the contour of the control target. The control target detector 111 may confirm the control target by using various means capable of identifying or specifying the control target.

The visual stimulus arrangement unit 112 may dispose the SSVEP-based visual stimulus STI flickering at the particular frequency on the virtual outline. In an embodiment, the visual stimulus arrangement unit 112 may dispose an additional visual stimulus providing an interface for controlling the control target 200 at a particular location that does not overlap the outline of the control target.

The electroencephalogram measurement module 120 may detect the electroencephalogram signal (EEG) through the electroencephalogram measurement headband (EHB) worn by the user. The electroencephalogram measurement module 120 may include a sensor unit 121 and an AFE 122.

The sensor unit 121 may sense the electroencephalogram signal measured at the electroencephalogram measurement headband (EHB). The AFE 122 may amplify the sensing electroencephalogram signal, and convert it to a digital signal.

The electroencephalogram measurement module 120 may be connected to the visual stimulus detection module 130 through a network.

The visual stimulus detection module 130 may include the EEG signal receiver 131, a visual stimulus signal detector 132, a feature extracting unit 133, and a visual stimulus classifier 134.

The electroencephalogram signal receiver 131 may receive the EEG signal from the sensor unit 121 of the electroencephalogram measurement module 120.

The visual stimulus signal detector 132 may analyze the received electroencephalogram signal to detect the visual stimulus signal (VEP) corresponding to the particular frequency. The visual stimulus signal detector 132 may perform preprocessing of the visual stimulus signal (SSVEP) for feature extraction.

The feature extracting unit 133 may extract a feature of the visual stimulus signal. The feature extracting unit 133 may extract a feature data for classification of visual stimuli among the preprocessed data.

The visual stimulus classifier 134 may classify the visual stimulus STI based on the extracted feature, and may identify the control target 200.

The control module 140 may perform control through the brain-computer interface (BCI).

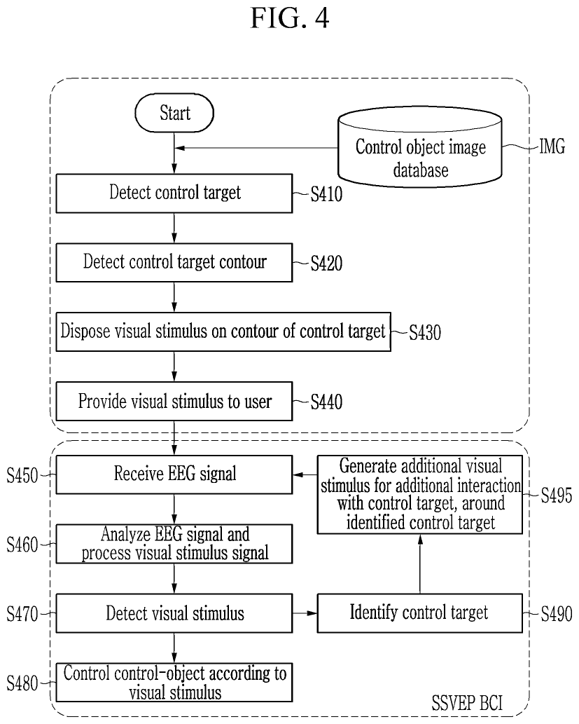

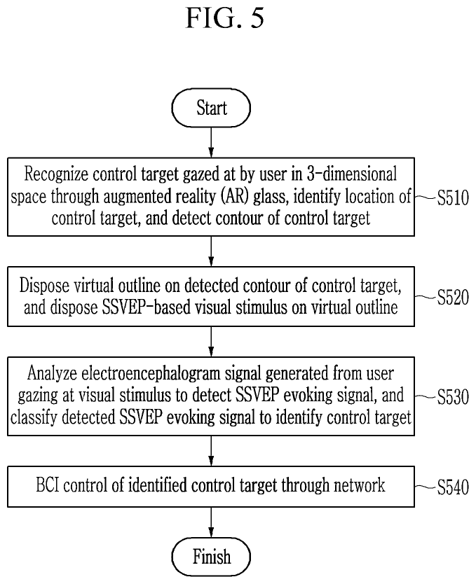

FIG. 4 and FIG. 5 are flowcharts showing a method for presenting a visual stimulus by using augmented reality according to an embodiment. A method for presenting a visual stimulus by using augmented reality of FIG. 4 and FIG. 5 may be performed through an apparatus for presenting a visual stimulus by using augmented reality 100 (refer to FIG. 1). Description will be made with reference to FIG. 1 to FIG. 3.

FIG. 4 shows steps (from step S410 to step S440) in which a visual stimulus presenting apparatus 100 synthesizes the visual stimulus STI through the augmented reality module 110 and steps (S450 to S495) in which the user controls the control target 200 through the visual stimulus STI.

In FIG. 4, the augmented reality module 110 may register control target image IMG from the database. At step S410, the augmented reality module 110 may detect the control target. Thereafter, at step S420, the augmented reality module 110 may detect location of the control target, and the contour of the control target.

Thereafter, at step S430, the augmented reality module 110 may dispose the virtual visual stimulus STI on the contour of the control target that appears as the AR glass AG. Thereafter, at step S440, the augmented reality module 110 may provide the disposed visual stimulus STI so as to be gazed at by the user.

Thereafter, at step S450, the visual stimulus detection module 130 may receive the EEG signal from the sensor unit 121. Thereafter, at step S460, the visual stimulus detection module 130 may analyze the EEG signal, and may perform feature extraction and classification of the visual stimulus signal. At step S470, the visual stimulus detection module 130 may detect a visual stimulus including the user's intention.

At step S480, the visual stimulus detection module 130 or the control module 140 may control the control target by using the detected visual stimulus. For example, a control module 130 may immediately perform on/off operation of the control target by using the detected visual stimulus.

At step S490, the visual stimulus detection module 130 may identify the control target based on the detected visual stimulus. Thereafter, at step S495, when an additional interaction is required, the augmented reality module 110 may generate a virtual additional visual stimulus so as not to overlap the control target around the identified control target.

Thereafter, the visual stimulus detection module 130 may receive the electroencephalogram signal of the user gazing at the virtual additional visual stimulus, process the visual stimulus signal obtained through the analysis to detect the visual stimulus, and control the control target based thereon.

In FIG. 5, at step S510, an apparatus 100 (hereinafter, a visual stimulus presenting apparatus) for presenting a visual stimulus by using augmented reality may recognize the control target gazed at by the user in a 3-dimensional space through the AR glass, identify a location of the control target, and detect the contour of the control target.

A visual stimulus presenting apparatus 100 may dispose the identifying means capable of identifying the control target on the detected control target. For example, at step S520, a visual stimulus presenting apparatus 100 may dispose the virtual outline on the contour of the control target, and may dispose the SSVEP-based visual stimulus on the virtual outline.

The SSVEP-based visual stimulus may be provided as a virtual image such as the checkerboard flickering or inverted at the particular frequency. The identifying means may include various means to confirm the control target in order to dispose the visual stimulus.

At step S530, a visual stimulus presenting apparatus 100 may analyze the electroencephalogram signal generated from the user gazing at the visual stimulus to detect as SSVEP evoking signal, and classify the detected SSVEP evoking signal to identify the control target.

At step S540, a visual stimulus presenting apparatus 100 may perform BCI control on the identified control target through a network.

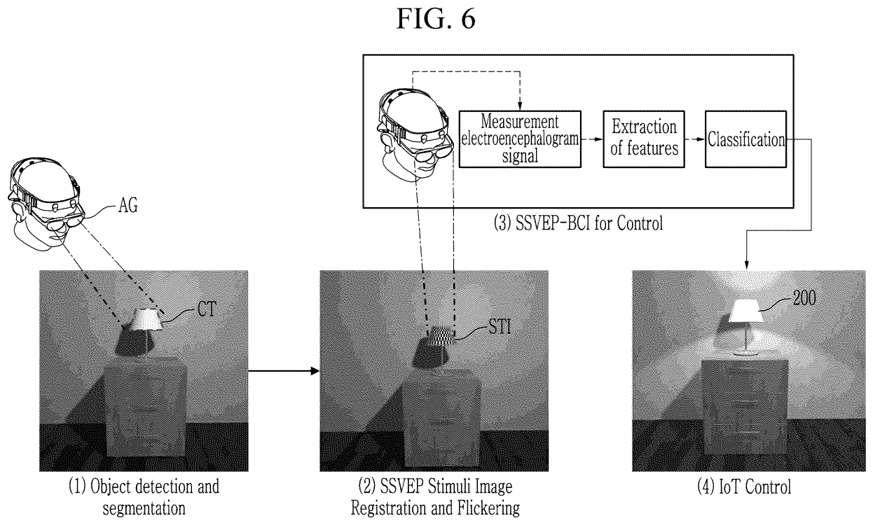

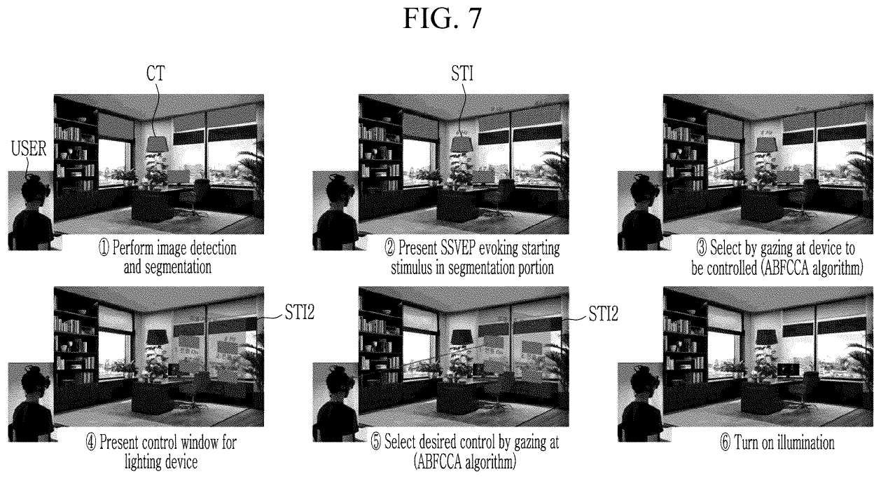

FIG. 6 and FIG. 7 are example diagrams showing the process of presenting and controlling the visual stimulus through a visual stimulus presenting method using augmented reality according to an embodiment of FIG. 4 and FIG. 5.

FIG. 6 is an example diagram showing a process of presenting and controlling a visual stimulus with respect to a single the control target according to an embodiment.

In FIG. 6, when the user wears the AG glass AG and gazes at an actual smart lighting device, a visual stimulus presenting apparatus 100 connected to the AG glass may recognize the lighting device, and may generate the virtual outline CT disposed on the lighting device. The smart lighting device may be an IoT device capable of communication with the visual stimulus presenting apparatus 100. Although FIG. 6 illustrates that the virtual outline CT is a 2-dimensional outline, the virtual outline CT may be a 3-dimensional outline that surrounds the lighting device in the actual 3-dimensional space.

Thereafter, the visual stimulus presenting apparatus 100 may generate a SSVEP visual stimulus STI on the virtual outline CT. The SSVEP visual stimulus STI may be synthesized on the lighting device in the same shape as the lighting device. The SSVEP visual stimulus STI may flicker at the particular frequency.

Thereafter, when the user gazes at the SSVEP visual stimulus STI, the visual stimulus presenting apparatus 100 may measure the electroencephalogram signal generated according to the frequencies, detect the visual stimulus STI through feature extraction and classification of the received steady-state visual evoked potential (SSVEP) based on the measured electroencephalogram signal, and identify the gazed control target 200 as a lighting device.

The visual stimulus presenting apparatus 100 may control the lighting device accordingly to the detected visual stimulus STI. For example, the visual stimulus presenting apparatus 100 may perform a BCI control, such as turning on or off the lighting device. When an additional control is required, the visual stimulus presenting apparatus 100 may generate the additional visual stimulus that provides interfaces around the lighting device.

FIG. 7 is an example diagram showing a process of presenting and controlling a visual stimulus with respect to a plurality of control targets according to an embodiment.

In FIG. 7, when the user wearing the AR glass gazes an actual 3-dimensional living space, the visual stimulus presenting apparatus 100 may recognize smart electronic devices capable of BCI control as the control targets. The visual stimulus presenting apparatus 100 may detect the location at which the recognized control targets is disposed, and may generate the virtual outline or the virtual images surrounding the control target (segmentation).

The visual stimulus presenting apparatus 100 may represent the SSVEP visual stimulus (or SSVEP evoked visual stimulus) STI on the virtual images disposed on the control targets. SSVEP visual stimuli STI disposed on the plurality of control targets may have each different frequency. That is, a plurality of SSVEP visual stimuli STI may flicker at different frequencies corresponding control targets, respectively.

The visual stimulus presenting apparatus 100 may detect a particular visual stimulus gazed at by the user, among a plurality of visual stimuli STI. For example, the visual stimulus presenting apparatus 100 may detect a particular visual stimulus gazed at by the user, among the plurality of visual stimuli STI, through the ABFCCA algorithm.

The visual stimulus presenting apparatus 100 may detect the frequency of a reference signal based on similarity between the visual stimulus signal detected from user gazing at visual stimulus of the particular frequency among the visual stimuli STI having different frequencies and a reference signal based on a predefined frequency, and may identify the control target gazed at by the user based on the detected frequency. Here, the method for analyzing the similarity of signals is not limited to a specific method and various methods may be used.

For example, the visual stimulus presenting apparatus 100 may analyze correlation between the visual stimulus signal detected from user gazing at visual stimulus of the particular frequency among the visual stimuli STI having different frequencies and a reference signal based on a predefined frequency to detect the frequency of the reference signal having a highest correlation, and may identify the control target gazed at by the user based on the detected frequency.

For example, when the user gazes at a visual stimulus disposed in a smart lighting device, the visual stimulus presenting apparatus 100 may detect a reference signal having a highest correlation with respect to the detected visual stimulus signal, detect a predefined frequency of the detected reference signal as a frequency of the visual stimulus gazed at by the user, and identify a lighting device that is the control target having corresponding frequency as the control target gazed at by the user.

Thereafter, the visual stimulus presenting apparatus 100 may generate a control window providing an interface for controlling the selected lighting device as an additional the visual stimulus STI2. The visual stimulus presenting apparatus 100 may present the additional the visual stimulus STI2 at a particular location that does not overlap the lighting device based on the location of the detected lighting device. When the user gazes at, in order to select, one option among a plurality of options appearing in the control window through the AG glass, the visual stimulus presenting apparatus 100 may perform a control with respect to the selected option.

For example, when the user gazes at a particular option for turning on the lighting device shown in the control window, the visual stimulus presenting apparatus 100 may detect the user's intention through analysis of the user's electroencephalogram and classification of the visual stimulus, and turn on the lighting device.

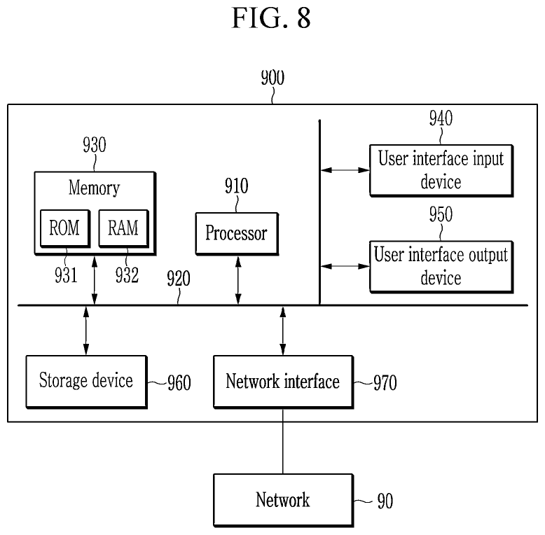

FIG. 8 is a drawing for explaining a computing device according to an embodiment.

Referring to FIG. 8, an apparatus and method for presenting a visual stimulus by using augmented reality according to an embodiment may be implemented by using a computing device 900.

The computing device 900 may include at least one of a processor 910, a memory 930, a user interface input device 940, a user interface output device 950 and a storage device 960 that communicate through a bus 920. The computing device 900 may also include a network interface 970 electrically connected to a network 90. The network interface 970 may transmit or receive signals with other entities through the network 90.

The processor 910 may be implemented in various types such as a micro controller unit (MCU), an application processor (AP), a central processing unit (CPU), a graphic processing unit (GPU), a neural processing unit (NPU), and the like, and may be any type of semiconductor device capable of executing instructions stored in the memory 930 or the storage device 960. The processor 910 may be configured to implement the functions and methods described above with respect to FIG. 1 to FIG. 7. For example, various elements described with reference to FIG. 1 to FIG. 7 may be implemented, as a whole or individually, by the processor 910 such that the processor 910 may be configured to implement the functions and methods described above.

The memory 930 and the storage device 960 may include various types of volatile or non-volatile storage media. For example, the memory may include read-only memory (ROM) 931 and a random-access memory (RAM) 932. In this embodiment, the memory 930 may be located inside or outside the processor 910, and the memory 930 may be connected to the processor 910 through various known means.

In some embodiments, at least some configurations or functions of the apparatus and method for presenting a visual stimulus by using augmented reality according to an embodiment may be implemented as a program or software executable by the computing device 900, and program or software may be stored in a computer-readable medium.

In some embodiments, at least some configurations or functions of the multi-input charging device apparatus and method for presenting a visual stimulus by using augmented reality according to an embodiment may be implemented by using hardware or circuitry of the computing device 900, or may also be implemented as separate hardware or circuitry that may be electrically connected to the computing device 900.

While this disclosure has been described in connection with what is presently considered to be practical embodiments, it is to be understood that the disclosure is not limited to the disclosed embodiments, but, on the contrary, is intended to cover various modifications and equivalent arrangements included within the spirit and scope of the appended claims.

What is claimed is:

1. An apparatus for presenting a visual stimulus by using augmented reality, comprising:
an augmented reality module configured to recognize a control target through an augmented reality (AR) glass, and to dispose the visual stimulus on the recognized control target;
an electroencephalogram measurement module configured to measure an electroencephalogram of a user gazing at the visual stimulus;
a visual stimulus detection module configured to analyze the measured electroencephalogram to detect a visual stimulus signal (VEP, and to process the detected visual stimulus signal to classify the visual stimulus and to identify the control target; and
a control module configured to control the identified control target through a network.

2. The apparatus of claim 1, wherein the augmented reality module is configured to register an image of the control target received from database.

3. The apparatus of claim 1, wherein the augmented reality module comprises a control target detector configured to detect a location of the control target gazed at by the user through the AR glass and to dispose a particular means to identify the control target.

4. The apparatus of claim 3, wherein the particular means comprises a virtual outline disposed on a contour of the control target.

5. The apparatus of claim 4, wherein the augmented reality module further comprises a visual stimulus arrangement unit configured to dispose a steady-state visual evoked potential (SSVEP)-based visual stimulus flickering at a particular frequency on the outline.

6. The apparatus of claim 5, wherein the visual stimulus arrangement unit is configured to dispose an additional visual stimulus that provides an interface for controlling the control target to a particular location that does not overlap the outline of the identified control target.

7. The apparatus of claim 1, wherein the visual stimulus detection module comprises:
an electroencephalogram signal receiver configured to receive an electroencephalogram signal;
a visual stimulus signal detector configured to analyze the received electroencephalogram signal to detect the visual stimulus signal corresponding to a particular frequency;
a feature extracting unit configured to extract a feature of the visual stimulus signal; and
a visual stimulus classifier configured to classify the visual stimulus based on the extracted feature and to identify the control target.

8. The apparatus of claim 1, wherein:
the augmented reality module is configured to detect locations of a plurality of control targets through the AR glass, dispose virtual outlines to contours of the plurality of control targets, respectively, and dispose VEP-based visual stimuli flickering at different frequencies on the virtual outlines, respectively; and
the virtual outlines comprises 3-dimensional outlines.

9. The apparatus of claim 8, wherein the visual stimulus detector is configured to analyze similarity between the visual stimulus signal detected from the electroencephalogram of the user gazing at the visual stimulus having a particular frequency and a reference signal according to a predefined frequency to detect a frequency having a highest similarity, and identify the control target gazed at by the user based on the detected frequency.

10. The apparatus of claim 8, wherein the visual stimulus detection module is configured to dispose an additional visual stimulus that provides an interface for an additional interaction after the control target is identified at a particular location that does not overlap the location of the control target.

11. A method for presenting a visual stimulus by using augmented reality, comprising:
recognizing a control target gazed at by a user through an augmented reality (AR) glass, and disposing the visual stimulus on the recognized control target;
measuring an electroencephalogram of the user gazing at the visual stimulus;
analyzing the measured electroencephalogram to detect a visual stimulus signal (VEP), and processing the detected visual stimulus signal to classify the visual stimulus and to identify the control target; and
controlling the identified control target through a network.

12. The method of claim 11, wherein the disposing the visual stimulus comprises detecting a location of the control target in a 3-dimensional space and disposing a virtual outline to a 3-dimensional contour of the control target.

13. The method of claim 12, wherein the disposing the visual stimulus further comprises disposing a steady-state visual evoked potential (SSVEP)-based visual stimulus flickering at a particular frequency on the virtual outline.

14. The method of claim 13, wherein the identifying the control target comprises:
extracting a feature of the visual stimulus signal; and
classifying the visual stimulus based on the extracted feature.

15. The method of claim 14, wherein the disposing the visual stimulus further comprises disposing an additional visual stimulus providing an interface for controlling the control target at a location that does not overlap the control target, when the control target is identified.

16. The method of claim 11, wherein the disposing the visual stimulus comprises detecting locations of a plurality of control targets in a 3-dimensional space to dispose a 3-dimensional virtual outline on each of contours of the plurality of control targets.

17. The method of claim 16, wherein the disposing the visual stimulus further comprises disposing the VEP-based visual stimulus flickering at different frequencies on the virtual outlines.

18. The method of claim 17, wherein the identifying the control target comprises analyzing similarity between a visual stimulus signal detected from the user gazing at the visual stimulus of a particular frequency visual stimuli having the different frequencies and a reference signal based on a predefined frequency to detect a frequency having a highest similarity, and identifying the control target gazed at by the user based on the detected frequency.

19. The method of claim 18, wherein the disposing the visual stimulus further comprises disposing an additional visual stimulus for controlling the identified control target on the 3-dimensional space so as not to overlap the identified control target.

20. The method of claim 11, wherein the visual stimulus comprises a checkerboard image inverted at a particular frequency.
